(19) United States
(12) Patent Application Publication
(10) Pub. No.: US 2026/0256728 A1
(43) Pub. Date: Sep. 3, 2026

(54) TREATMENT FOR ASYMPTOMATIC STARGARDT DISEASE PATIENTS

(71) Applicant: Alkeus Pharmaceuticals, Inc., Cambridge, MA (US)

(72) Inventor: Leonide C. Saad, Cambridge, MA (US)

(21) Appl. No.: 19/656,478

(22) Filed: Apr. 23, 2026

Related U.S. Application Data
(63) Continuation of application No. PCT/US2024/062408, filed on Dec. 31, 2024.
(60) Provisional application No. 63/617,005, filed on Jan. 2, 2024.

(51) Int. Cl. A61K 31/232; A61P 27/02

(52) U.S. Cl. CPC A61K 31/232; A61P 27/02

(57) ABSTRACT

Disclosed is a method of delaying the onset of Stargardt disease symptoms in an asymptomatic Stargardt disease patient. Also disclosed is a method of delaying the onset of vision loss in an asymptomatic Stargardt disease patient. Also disclosed is a method of treating a Stargardt disease patient to delay the onset of vision loss in the Stargardt disease patient, wherein treatment is initiated prior to the patient experiencing vision loss resulting from the Stargardt disease. The methods comprise administering to the patient an effective amount of gildeuretinol acetate.

### CROSS-REFERENCE TO RELATED APPLICATIONS

This application is a continuation of and claims benefit under 35 U.S.C. § 120 from PCT application PCT/US2024/062408, filed on Dec. 31, 2024, which application claims the benefit of priority of U.S. Provisional Application No. 63/617,005, filed on Jan. 2, 2024, the content of which is incorporated by reference in its entirety herein.

### FEDERALLY SPONSORED RESEARCH OR DEVELOPMENT

This invention was made with government support under Grants No. FD004098 and FD006316 awarded by the Food and Drug Administration. The government has certain rights in the invention.

### BACKGROUND OF THE INVENTION

Stargardt disease is a genetic cause of blindness in children and young adults. Stargardt disease typically presents within the first two decades of life, even though symptoms can appear during adulthood and as late as the seventh decade of life. It is usually characterized by a progressive loss of central vision causing blurry vision and, occasionally, an increasing difficulty in adapting to the dark. Most affected individuals also have impaired color vision. Photophobia may be present. Symptoms of vision loss due to Stargardt disease continue to worsen over time, and cannot be corrected by glasses or contact lenses, because the light sensitive part of the eye, the retina, is damaged by the disease.

Although there is currently no approved treatment for Stargardt disease, it has been proposed that gildeuretinol acetate, a form of deuterated vitamin A acetate, could be used as a method to treat macular degeneration (RE 47,045). The structure of gildeuretinol acetate is shown below:

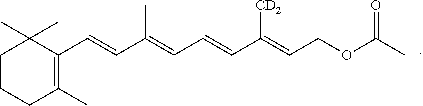

As with retinol acetate (vitamin A acetate), when consumed, the body converts gildeuretinol acetate between its three primary forms (C₂₀D₃-retinyl esters, C₂₀D₃-retinol, and C₂₀D₃-retinaldehyde). C₂₀D₃-retinol is also referred to herein as “gildeuretinol” and C₂₀D₃-retinol acetate as “gildeuretinol acetate”.

It would be highly desirable to develop treatments to slow the onset or even prevent the onset of impaired vision in Stargardt disease patients.

### SUMMARY OF THE INVENTION

It has now been found in a clinical trial that treatment with gildeuretinol acetate slowed the expansion of retinal atrophic lesions in symptomatic Stargardt disease patients (see Example 1).

Surprisingly, it was found that treatment with gildeuretinol acetate completely prevented vision loss in asymptomatic Stargardt disease patients over the duration of the clinical trial (see Example 2). In the absence of treatment, it was expected that these asymptomatic Stargardt disease patients would show rapid disease progression. This was further expected because these asymptomatic Stargardt disease patients had Stargardt disease 2-year older siblings who already were showing symptoms of vision loss.

Surprisingly, during the 2-4 year treatment period of the clinical trial, no visible disease progression by fundus autofluorescence imaging occurred, no retinal flecks developed, no changes in hyper- or hypo-autofluorescence were observed in the asymptomatic patients treated with gildeuretinol acetate. In contrast, the siblings with Stargardt disease who had not taken gildeuretinol acetate, when age-matched, presented with retinal flecks, hyper- or hypo-autofluorescence, regions of retinal atrophy, which continued to worsen year-over-year. Furthermore, visual acuity remained at approximately 20/20 or better in the treated asymptomatic patients. In contrast, their untreated age-matched siblings lost significant vision (Example 2 and FIG. 3). Based in part on these discoveries, methods of treating asymptomatic Stargardt disease patients with gildeuretinol acetate to suppress, prevent or delay the onset of vision loss associated with Stargardt disease or suppress, prevent or delay the onset of symptoms associated with Stargardt disease are disclosed herein.

One embodiment of the invention is a method of delaying the onset of Stargardt disease symptoms in an asymptomatic Stargardt disease patient. The method comprises administering to the patient an effective amount of gildeuretinol acetate.

Another embodiment of the invention is a method of delaying the onset of vision loss in an asymptomatic Stargardt disease patient. The method comprises administering to the patient an effective amount of gildeuretinol acetate.

Another embodiment of the invention is a method of treating a Stargardt disease patient to delay the onset of vision loss in the Stargardt disease patient, wherein treatment is initiated prior to the patient experiencing vision loss resulting from the Stargardt disease. The method comprises administering to the patient an effective amount of gildeuretinol acetate.

Another embodiment of the invention is a method of delaying the onset of symptoms in an asymptomatic patient with biallelic Stargardt Associated Mutations, comprising administering to the patient an effective amount of gildeuretinol acetate.

Another embodiment of the invention is a method of delaying the onset of vision loss in an asymptomatic patient with biallelic Stargardt Associated Mutations, comprising administering to the patient an effective amount of gildeuretinol acetate.

Another embodiment of the invention is a method of treating a patient with biallelic Stargardt Associated Mutations, to delay the onset of vision loss in the patient, wherein treatment is initiated prior to the patient experiencing vision loss, comprising administering to the patient an effective amount of gildeuretinol acetate.

Another embodiment of the invention is a method of preventing the symptoms of Stargardt disease in a patient susceptible to Stargardt disease or likely to experience symptoms of Stargardt disease, comprising administering to the patient an effective amount of gildeuretinol acetate prior to the patient experiencing symptoms of Stargardt disease.

Another embodiment of the invention is gildeuretinol acetate for delaying the onset of Stargardt disease symptoms in an asymptomatic Stargardt disease patient.

Another embodiment of the invention is gildeuretinol acetate for delaying the onset of vision loss in an asymptomatic Stargardt disease patient.

Another embodiment of the invention is gildeuretinol acetate for treating a Stargardt disease patient to delay the onset of vision loss in the Stargardt disease patient, wherein treatment is initiated prior to the patient experiencing vision loss resulting from the Stargardt disease.

Another embodiment of the invention is the use of gildeuretinol acetate for the manufacture of a medicament for delaying the onset of Stargardt disease symptoms in an asymptomatic Stargardt disease patient.

Another embodiment of the invention is the use of gildeuretinol acetate for the manufacture of a medicament for delaying the onset of vision loss in an asymptomatic Stargardt disease patient.

Another embodiment of the invention is the use of gildeuretinol acetate for the manufacture of a medicament for treating a Stargardt disease patient to delay the onset of vision loss in the Stargardt disease patient, wherein treatment is initiated prior to the patient experiencing vision loss resulting from the Stargardt disease.

### BRIEF DESCRIPTION OF THE FIGURES

FIG. 1. is a graph showing the percent gildeuretinol (deuterated vitamin A) versus total plasma vitamin A (as gildeuretinol+retinol) over time in months in patients administered 14 mg/day gildeuretinol acetate; in patients administered 24 mg/day gildeuretinol acetate for 12 months followed by 14 mg/day gildeuretinol acetate for twelve months; in patients administered placebo for 12 months followed by 14 mg/day gildeuretinol acetate for 12 months; and in patients administered placebo for 24 months.

FIG. 2 is a graph showing the growth in size of atrophic retinal lesions over time in patients treated with gildeuretinol acetate (ALK-001) compared with placebo. Dotted line, untreated arm (natural history+placebo): growth rate of 0.24 mm/year; 15 patients taking placebo and 54 natural history cases. Continuous line, gildeuretinol acetate arm: growth rate of 0.19 mm/year; 34 patients taking gildeuretinol acetate. Error bars are 95% confidence intervals.

FIG. 3 is a graph showing the age-matched visual acuity of three asymptomatic Stargardt disease patients, taking gildeuretinol acetate 14 mg daily (bold curves) alongside their untreated Stargardt disease siblings. Each symbol represents a visual acuity measurement for each eye. Curves show average visual acuity for both eyes of each patient. Dotted arrows show comparisons between siblings.

FIG. 4 is a graph showing mean retinal sensitivity (dB) over time in three asymptomatic Stargardt disease patients taking gildeuretinol acetate 14 mg daily. Lines show mean retinal sensitivity of both eyes. Lines are piecewise linear regressions with a knot time of 12 months. Each symbol represents a retinal sensitivity measurement for each eye. Patient 1 retinal sensitivity was measured with a Nidek MP-1 (Nidek, Gamagori, Japan). Patients 2 and 3 retinal sensitivity was measured with the Center Vue Macular Integrity Assessment or MAIA (CenterVue, Padova, Italy). To plot Patient 1 on a comparable scale, 4 dB was added to all measured thresholds of Patient 1.

FIG. 5 is a graph showing retinal photoreceptor loss over time in three asymptomatic Stargardt disease patients taking gildeuretinol acetate 14 mg daily. Lines are piecewise linear regressions with a knot time of 6 months and show the mean area of retinal photoreceptor loss of both eyes. Each symbol represents the area of photoreceptor loss for one eye. To measure photoreceptor loss, the ellipsoid zone layer was evaluated on b-scans acquired on Heidelberg engineering optical coherence tomography (Heidelberg, Germany).

### DETAILED DESCRIPTION

Disclosed herein are methods of treating asymptomatic Stargardt disease patients with gildeuretinol acetate to suppress, prevent or delay the onset of vision loss associated with Stargardt disease or to suppress, prevent or delay the onset of symptoms associated with Stargardt disease.

Gildeuretinol acetate is retinol acetate in which the three hydrogen atoms at carbon twenty have been replaced or enriched with deuterium, a stable isotope of hydrogen. The structure of gildeuretinol acetate is shown below:

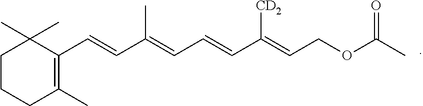

Gildeuretinol acetate is also referred to as “C₂₀D₃-retinol acetate”. The deuterium enrichment in gildeuretinol acetate is at least 90.0%; at least 95.0%; at least 97.0%; or at least 98.0%. For example, the deuterium enrichment is from 90.0% to 99.8%; 95.0% to 99.8%; 97.0% to 99.8%; 98.0% to 99.8%; 90.0% to 99.5%; 95.0% to 99.5%; 97.0% to 99.5%; or 98.0% to 99.5%. The deuterium enrichment in the gildeuretinol acetate used in Examples 1-3 was from 95.0% to 99.5%. “Deuterium enrichment” for gildeuretinol acetate is a mole percent and is determined by dividing the number of deuterium atoms at the C₂₀ position by the total number of hydrogen H (as protium)+deuterium atoms at the C₂₀ position. Deuterium enrichment can be determined for example by mass spectrometry from the intensities of the 269.2 molecular ion peak (C₂₀H₃-vitamin A), the 270.2 molecular ion peak (C₂₀H₂D-vitamin A), the 271.2 molecular ion peak (C₂₀HD₂-vitamin A) and the 272.2 (C₂₀D₃-vitamin A) molecular ion peak.

Suitable formulations for gildeuretinol acetate are as described in U.S. RE47,045, the entire teachings of which are incorporated herein by reference. Gildeuretinol acetate can be administered by any suitable means, including orally, subcutaneously, parenterally, intravenously or by injection, topically (including for example as an eyedrop, implant, drug-eluting or other device, intravitreal, suprachoroidal or other injections directly into the eye), but is typically administered orally, as a pill, for example a tablet, a capsule or a softgel. In one aspect, the gildeuretinol acetate is administered orally as a pill. In one aspect, the pill is taken daily. In one aspect, the pill comprises gildeuretinol acetate dissolved in soybean oil and stabilized with an antioxidant. In one example, the antioxidant is butylated hydroxytoluene comprising about 0.1% to 0.2% by weight of the oil. In another aspect, about 10 to 24 mg of gildeuretinol acetate is dissolved into about 400 mg of the oil.

“Stargardt disease” or “Stargardt” as used herein, refers to the diagnosis made by a physician of a patient. Synonyms of Stargardt disease include, without limitation, Stargardt juvenile macular degeneration, Stargardt juvenile macular dystrophy, Stargardt inherited macular degeneration, juvenile macular degeneration and fundus flavimaculatus.

“Stargardt disease patient” (or “patient”) refers to a human with Stargardt disease.

“Symptomatic Stargardt disease patient” refers to a Stargardt disease patient who has developed one or more symptoms of Stargardt disease.

“Asymptomatic Stargardt disease patient” refers to a Stargardt disease patient who does not have any symptoms of Stargardt disease.

A “Stargardt disease symptom”, or “symptom” as used herein, refers to a characteristic or feature of Stargardt disease that can be perceived or identified by the patient. In one example, a Stargardt disease symptom is some measurable degree of vision loss. The vision loss associated with Stargardt disease is characterized by loss of central vision, flashes of light, blurred vision, difficulty in seeing in dim lighting or at night, difficulty in distinguishing colors, difficulty in adjusting to bright light exposure, difficulty in adjusting to the dark, difficulty tracking moving objects, difficulty in reading, writing, or recognizing faces due to vision loss, and blind spots in the field of vision. Involuntary eye movements are also a symptom of Stargardt disease.

A patient with “asymptomatic” Stargardt disease, as it is used herein, refers to a patient with Stargardt disease who does not yet have any of the symptoms of Stargardt disease.

The term “symptom”, as it is used herein, does not include those characteristics or features of Stargardt disease that can be measured or identified by a physician or other medical professional but cannot be perceived or identified by the patient. Such characteristics and features of Stargardt disease that are measurable or identifiable only by a physician or other medical professional but not by the patient are referred to herein as “Diagnostic Indicators” of Stargardt disease. Often times, Diagnostic Indicators precede the appearance of symptoms. Therefore, a person who is asymptomatic with respect to Stargardt disease can be diagnosed with Stargardt disease if one or more of these Diagnostic Indicators are present. The presence of these Diagnostic Indicators is in one aspect identified by visually examining the eye with the aid of a slit lamp or similar imaging device. Imaging devices that image the retina, such as fluorescein angiography (FA), fundus autofluorescence (FAF), color fundus photography (CFP), and optical coherence tomography (OCT) can also identify Diagnostic Indicators of Stargardt disease. Examples of Diagnostic Indicators include retinal atrophic lesions, retinal flecks, pigment migration, retinal thinning, ellipsoid zone loss, abnormal or thickened external limiting membrane, photoreceptor loss, retinal atrophy, areas of hypo and hyper-autofluorescence and maculopathy. Often times, Diagnostic Indicators first appear in the parafoveal region. Retinal atrophic lesions, flecks and other Diagnostic Indicators are also present when Stargardt disease becomes symptomatic and vision loss occurs. The size and/or growth of retinal atrophic regions and flecks can be used as a measure of disease progression.

Stargardt disease is also associated with certain genetic abnormalities known as “mutations” or “variants”. The most common example of a mutation that causes Stargardt disease is when one or more mutations are found in the ATP binding cassette subfamily A member 4 gene (ABCA4), which is a gene that encodes a membrane-associated protein belonging to the superfamily of ATP-binding cassette (ABC) transporters. ABC proteins transport various molecules across extra- and intracellular membranes. ABCA4 mutations that are associated with retinal degeneration and Stargardt disease can be found in databases, including The Genome Aggregation Database (gnomAD), the Leiden Open Variation Database (LOVD), ClinVar, the Exome Aggregation Consortium (ExAC) and The Human Gene Mutation Database (HGMD®). In one aspect, the Stargardt disease patient being treated according to the disclosed method has an ABCA4 mutation.

A mutation or variant that is associated with Stargardt disease, such as a mutation in the ABCA4 gene is referred to herein as a “Stargardt Associated Mutation”. Not all mutations are pathogenic (or disease-causing). A “pathogenic mutation” or “disease-causing mutation”, as used herein, is a mutation determined to be responsible for the Stargardt disease in a patient, or is being used to diagnose Stargardt disease in a patient, or is one allowing a physician to make a treatment decision of a patient. Pathogenicity of novel mutations can be evaluated using a variety of criteria including for example the Standards and Guidelines for the Interpretation of Sequence Variants from the American College of Medical Genetics and Genomics (ACMG) or the Association for Molecular Pathology.

“Biallelic”, as used herein, shall designate a patient who has two or more mutations.

Asymptomatic Stargardt patients can be identified through routine screening of patients for a Diagnostic Indicator and/or a Stargardt Associated Mutation. Treatment with gildeuretinol acetate, as described herein, can be initiated once a patient is identified as having one or more Diagnostic Indicators and/or a Stargardt Associated Mutation. Therefore, one embodiment of the invention is a method of identifying and then treating a subject with asymptomatic Stargardt disease. The method comprises screening a patient for a Diagnostic Indicator(s) (e.g., retinal atrophic lesions, retinal flecks, pigment migration, retinal thinning, ellipsoid zone loss, abnormal or thickened external limiting membrane, photoreceptor loss, retinal atrophy, areas of hypo and hyper-autofluorescence and maculopathy or a Stargardt Associated Mutation (e.g., one or more ABCA4 mutation(s)). If the asymptomatic patient has a Diagnostic Indicator for Stargardt disease or a Stargardt Associated Mutation, the patient is administered an effective amount of gildeuretinol acetate.

Alternatively, treatment with gildeuretinol acetate can be delayed up until the time the asymptomatic patient is identified as having both a Diagnostic Indicator (e.g., retinal atrophic lesions, retinal flecks, pigment migration, retinal thinning, ellipsoid zone loss, abnormal or thickened external limiting membrane, photoreceptor loss, retinal atrophy, areas of hypo and hyper-autofluorescence and maculopathy and a Stargardt Associated Mutation (e.g., ABCA4 mutation). In another aspect of identifying and then treating a subject with asymptomatic Stargardt disease, the patient is assessed for a Diagnostic Indicator and a Stargardt Associated Mutation. If the patient is identified as having both a Stargardt Associated Mutation and a Diagnostic Indicator, the patient is treated with an effective amount of gildeuretinol acetate. If the patient is identified as having a Stargardt Associated Mutation but not a Diagnostic Indicator, monitoring of the patient may also be continued up until the time the presence of a Diagnostic Indicator is detected and treatment with gildeuretinol acetate is then begun. Patients with a Stargardt Associated Mutation who do not have a Diagnostic Indicator should be routinely monitored for the development of a Diagnostic Indicator and the disclosed gildeuretinol acetate treatment initiated before the onset of Stargardt disease symptoms. As shown in the Example 2, the onset of Stargardt disease symptoms can be prevented at least for significant periods of time if treatment is begun when the patient is asymptomatic. Patients who are at risk of Stargardt disease, i.e., patients with a family history of Stargardt disease or a family history of having a Stargardt Associated Mutation, should be screened for Stargardt Associated Mutation and/or Diagnostic Indicators at an early age before the onset of symptoms.

In yet another aspect of identifying and then treating a subject with asymptomatic Stargardt disease, the asymptomatic patient is screened for the presence of a Diagnostic Indicator. If the patient is identified as having a Diagnostic Indicator, the patient is then assessed for a Stargardt Associated Mutation to confirm the diagnosis of Stargardt disease. If the patient has a Stargardt Associated Mutation, treatment with an effective amount of gildeuretinol acetate is then begun.

Genotyping technologies for detecting a Stargardt Associated Mutation include hybridization and/or PCR-based methods, such as iPLEX, pyrosequencing, microarray, and next-generation sequencing. The selection of genotyping techniques depends on parameters such as the amount (i.e., the number of genetic markers) and type (i.e., precision) of genetic information needed, the size of the cohort (i.e., the number of individuals), the computational capacity available (e.g., pre-processing, post-processing), and the financial considerations. ABCA4 mutations that are associated with retinal degeneration can be found in databases, including The Genome Aggregation Database (gnomAD), the Leiden Open Variation Database (LOVD), ClinVar, the Exome Aggregation Consortium (ExAC) and The Human Gene Mutation Database (HGMD®). Preferably, the genotyping should be able to detect non-coding variants deeper than +20 base pairs from the exon-intron boundary. These genotyping methods make use of DNA sampled from the potential patient, including blood, saliva, hair, or paraffin-embedded biopsy tissues. Methods for DNA isolation have been described. DNA isolation from the biological samples involves extracting chromosomal DNA from the cellular nuclei and removing proteins and cell debris to yield a DNA sample.

Gildeuretinol acetate can be prepared according to methods described in U.S. RE47,045, and the following references include procedures that can be useful in preparing gildeuretinol: Pardoen et al., Can. J. Chem. 63:1431 (1985); Bonrath et al., Org. Process Res. Dev. 27:1557 (2023); and Bergen et al., Journal of Labelled Compounds XXV: 11 (1987).

The dosage amount will vary with the route of administration, the rate of excretion, the duration of the treatment, the identity of any other drugs being administered, the age and size of the patient. In general, a suitable dose of gildeuretinol acetate will be the lowest dose effective to produce the desired effect, e.g., about 0.1 to about 90 mg/day to a patient. The effective dose of a compound may be administered as two, three, four, five, six or more sub-doses, administered separately at appropriate intervals throughout the day, or possibly less frequently, for example once every other day, once a week, once every other week, once a month. In one embodiment, 10-35 mg are administered once daily, e.g., orally. Alternatively, 14 mg or 24 mg are administered once daily, e.g., orally.

The following Examples are offered to illustrate exemplary embodiments of the invention and do not define or limit its scope.

### EXEMPLIFICATION

EXAMPLE 1: Administering daily doses of gildeuretinol acetate ranging from approximately 14 to 24 mg of gildeuretinol acetate replaces more than 80% of the serum vitamin A with gildeuretinol within a matter of weeks. 24 months of daily gildeuretinol acetate at 14 to 24 mg slows retinal degeneration in symptomatic Stargardt disease patients.

### Methods

### Study Design

The Tolerability and Effects of gildeuretinol acetate in Stargardt Disease (Clinical trial group 1, also known as TEASE-1) was a 24-month, multicenter, double-masked, placebo-controlled trial designed to evaluate the efficacy and safety of daily oral gildeuretinol acetate in slowing the growth of atrophic retinal lesions in Stargardt disease patients. The trial was conducted at multiple sites in the United States (NCT02402660).

The study protocol was approved by independent institutional review boards. The trial was conducted in accordance with the principles of the Declaration of Helsinki, International Conference on Harmonization E6 Good Clinical Practice Consolidated Guideline, and other relevant regulations. All participants provided written informed consent prior to screening or initiation of any study-related procedures and received visit-based reimbursement as approved by the institutional review boards. No additional stipends or incentives were provided.

### Patients

Patients 12 years or older, clinically diagnosed with Stargardt disease, with at least one ABCA4 mutation, at least one eye presenting with well-demarcated areas of reduced fundus autofluorescence including retinal atrophic lesions, and with any visual acuity, were enrolled.

### Randomization, Masking, and Treatments

Patients were randomized to gildeuretinol acetate 14 mg capsules, gildeuretinol acetate 24 mg capsules, or placebo capsules, to be taken once daily for one year. After one year, 50% of randomly selected patients receiving placebo were crossed over in a 1:1 ratio to gildeuretinol acetate 14 mg capsules or gildeuretinol acetate 24 mg capsules for a second year. If a masked interim review of plasma gildeuretinol levels indicated that the 14 mg and 24 mg doses achieved the same average percentage replacement of plasma vitamin A, patients initially randomized to the 24 mg dose received the 14 mg dose during the second year.

Gildeuretinol acetate and placebo capsules were identical appearing capsules. Patients were advised to avoid vitamin A-containing supplements and liver-based products. Fruits and vegetables could be consumed without limitation. Trial visits occurred at 8 weeks and 6, 12, 18, and 24 months following screening and randomization.

### Natural History Cases

Deidentified retinal images of patients not enrolled in the trial were included as a natural history cohort. This natural history cohort comprised patients diagnosed with Stargardt disease of any age, with two or more longitudinal fundus autofluorescence images taken at least four months apart over a maximum period of 2.5 years.

### Primary Efficacy Endpoint

The prespecified primary efficacy endpoint was the growth rate of the square root area of atrophic retinal lesions measured by fundus autofluorescence retinal imaging comparing gildeuretinol acetate versus the untreated arm (placebo combined with natural history). Only well-delineated and homogeneous retinal atrophic lesions ≥0.2 mm² with a summed area ≥0.5 mm² were included in the primary analysis.

### Statistical Analysis

The primary endpoint analysis used a linear mixed-effect model (LMEM) with repeated measures, conferring a higher power to detect smaller effects. All observed data, including both eyes, were utilized. Treatment duration was computed as a continuous measure for each visit based on years since randomization. Adjustments for baseline retinal atrophy used the baseline atrophic retinal lesion size as a fixed effect in the LMEM. Additional fixed effects included treatment arm, treatment duration, and interaction between the treatment arm and treatment duration. Random effects included patient, eye, grader to adjust for correlation between eyes within a patient, graders' assessment of an eye, and observations of the same eye over time. A random effect slope term was added to the effect of treatment duration. Patients who crossed over to gildeuretinol acetate after year one were included in the untreated arm from baseline to year one, then in the gildeuretinol acetate arm from baseline to year one upon crossing over to gildeuretinol acetate. The full analysis set (FAS) comprised all randomized patients who received at least one dose of the study drug.

Growth rates of atrophic retinal lesions were computed as two segmented lines over the 24-month treatment period; the first segment was between 0 and 6 months, and the second was between 6 and 24 months. All available data between 0 and 24 months was used to compute the slope of each segment. The primary efficacy endpoint was the slope of the second segment. No constraints were imposed on the Y-intercept. All growth rates were calculated on a per-year basis.

No imputations were made for missing data because the LMEM yields valid inferences under the assumption of a missing-at-random mechanism. The two-sided P-value of the primary efficacy endpoint was calculated using Kenward-Roger's method for handling denominator degrees of freedom and small samples. The goodness of fit of the linear mixed-effect model was provided as R-squared (R²). Analyses were conducted in R (R Core Team, 2020). Prespecified, unadjusted sensitivity analyses of the primary efficacy endpoint included growth rates of untransformed areas of retinal atrophy and growth rates using a single-slope from baseline until the last available visit. Post-hoc unadjusted analyses were performed to evaluate the potential contribution of patient characteristics.

### Results

The trial was conducted between August 2015 and December 2020. Fifty-three patients were screened, and 50 were randomized to gildeuretinol acetate 14 mg (n=15), gildeuretinol acetate 24 mg (n=15) or placebo (n=20). After one year, 10 placebo patients switched to gildeuretinol acetate. Four patients dropped out, unrelated to treatment: three from the gildeuretinol 14 mg group and one from the placebo group. All collected data from dropouts were utilized. Seven patients had summed atrophic retinal lesions smaller than <0.5 mm² and thus were excluded from analysis as specified in the statistical analysis plan: three from the gildeuretinol group acetate, three from the crossover group, and one from the placebo group. One patient in the placebo group was excluded because they had no follow-up data.

A total of 63 natural history cases were received from five centers. One case was excluded because the patient was randomized in the trial, one had a PRPH2 mutation, one had histoplasmosis, and one had atrophic lesions outside the imaging field. Four cases were excluded because they lacked follow-up within 4 months, and one because it had a follow-up visit over 2.5 years apart.

The primary efficacy analysis included 96 patients: 42 randomized patients and 54 natural history cases. Overall, demographic and clinical characteristics at baseline were similar in the two groups. The median age was 55, and 56% of patients were female. Of the randomized patients, 41 had two or more ABCA4 mutations, and one had one ABCA4 mutation. Of the natural history cases, 47 had two or more ABCA4 mutations, 3 had one mutation, and genotyping was unavailable for 7. Three to seven time points for at least one eye were available for 39 cases, while 16 cases had two time points for at least one eye.

### Plasma Gildeuretinol

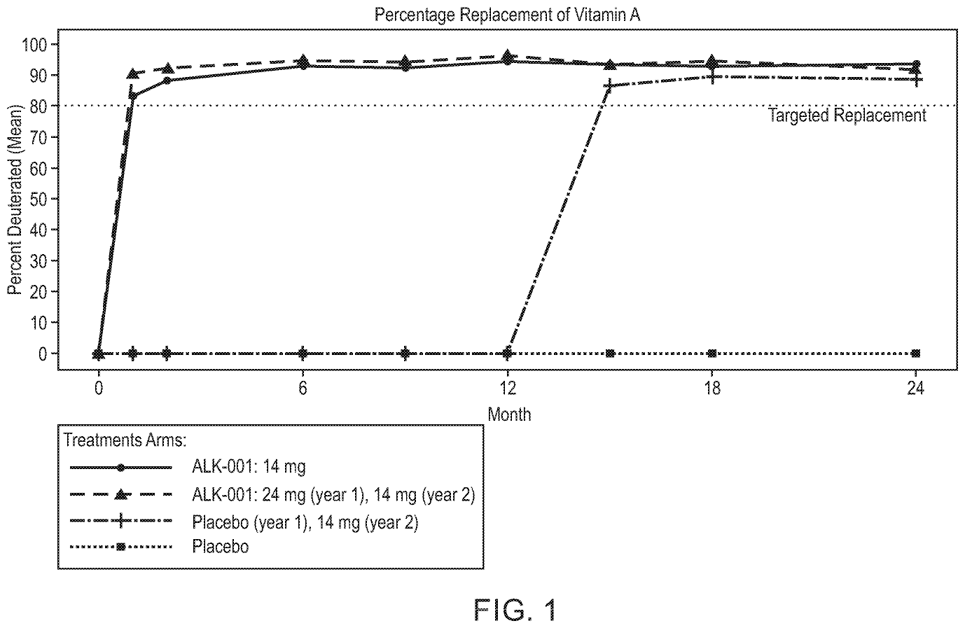

In the 8-week pharmacokinetic analysis, the percentage of gildeuretinol over total vitamin A (as retinol+gildeuretinol) in the 14 mg and 24 mg cohorts was equivalent (87% and 92%, respectively). Thus, all treated patients received gildeuretinol acetate 14 mg for the second year of treatment, and patients taking any dose formed the gildeuretinol acetate arm. A greater than 80% replacement was sustained throughout the treatment period. After six months, over 80% of plasma vitamin A was replaced in 39 out of 40 patients receiving gildeuretinol acetate (FIG. 1). One crossover patient did not achieve 80% replacement due to non-compliance. Total plasma vitamin A and retinoic acid, the combined vitamin A and gildeuretinol-derived forms, were the same between gildeuretinol acetate and placebo groups. No patients receiving placebo had detectable levels of gildeuretinol.

### Primary Endpoint

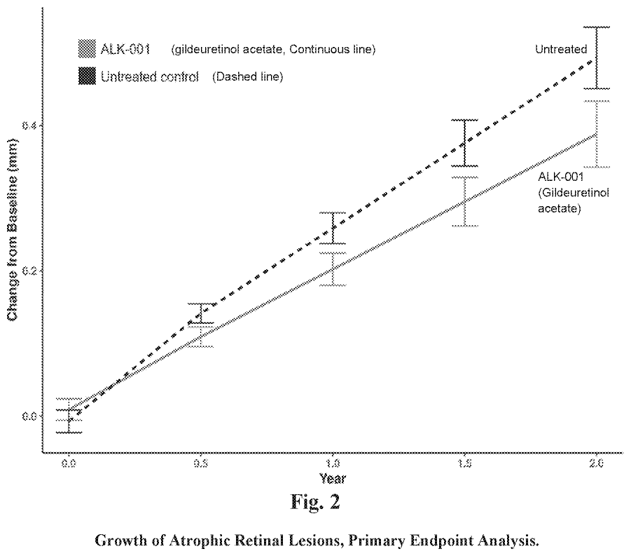

The growth rate of atrophic retinal lesions was 0.19 mm/year with gildeuretinol acetate and 0.24 mm/year with control, a difference of 0.05 mm/year (95% confidence interval [CI], 0.03 to 0.07), corresponding to a relative 21% slower growth rate in patients treated with gildeuretinol acetate (P<0.001, R²=0.9996) (FIG. 2).

The significant positive treatment effect persisted across different calculations for estimating the growth rate of atrophic retinal lesions and remained consistent when considering various scenarios, such as excluding the entire natural history cohort, including or excluding natural history cases with two-time points, cases with a single identified ABCA4 mutation, or cases with no genetic information (Table 1).

TABLE 1
Estimated Treatment Effects Using Different Calculations
and Populations Estimation Methods and/Population

| | Percentage |
|---|---|
| | Slowing of |
| | Atrophic Retinal |
| | Lesion Growth |
| | Rate With |
| Estimation Method/Population | Gildeuretinol |
| Using untransformed areas of atrophic | 28% |
| lesions (mm²) | |
| Using a single-slope from 0 to 24 months (mm²) | 31% |
| Crossover patients: 12 months placebo vs. | 21% |
| 12 months treatment | |
| Excluding 13 natural history cases without | 20% |
| any confirmed ABCA4 mutations and 6 natural | |
| histories with one confirmed mutation | |
| Excluding 2 natural history cases <18 years | 21% |
| old, 9 natural histories >60 years old | |
| Excluding 16 natural histories with only two | 20% |
| follow up time points | |
| Excluding natural history, randomized | 23% |
| patients, only, two slopes (mm²) | |
| Excluding natural history, randomized | 30% |
| patients only, one slope (mm²) | |
| Excluding 3 randomized patients with one | 20% |
| confirmed mutation | |

Gildeuretinol acetate met its prespecified primary efficacy endpoint, slowing the mean growth rate of atrophic retinal lesions relative to untreated patients composed of placebo and natural history.

### Safety

No serious adverse events were associated with gildeuretinol acetate and no patient discontinued treatment due to side effects. All adverse events were mild or moderate, and no association was observed between treatment groups and the frequency of adverse events (Table 2). No changes in visual acuity were observed. By physical examination, electrocardiogram, vital signs, hematology, biochemistry and lipid panels, no changes were attributed to gildeuretinol acetate. Blurred optic disc margins were observed in one patient receiving the 24 mg dose upon ocular exam at the 12-month visit, associated with complaints of transient visual obscuration. Elevated intracranial pressure was measured, and papilledema Grade 1 (modified Frisen scale) was diagnosed. This event resolved upon temporary interruption of gildeuretinol acetate and treatment with acetazolamide. The patient resumed treatment and completed the trial without recurrence.

TABLE 2
Adverse events observed in the TEASE-1 trial, presented by MedDRA System Organ Class and
Preferred Term. Coded using MedDRA version 22.0. Interim data as of December 2020.

| | | Number of patients experiencing event (%) | | | |
|---|---|---|---|---|---|
| | | gildeuretinol | gildeuretinol | | |
| System Organ | | acetate 14 mg | acetate 24 mg | Crossover | Placebo |
| Class | Preferred Term | (N = 15) | (N = 15) | (N = 10) | (N = 10) |
| Overall | Overall | 12 (80) | 13 (86.7) | 8 (80) | 9 (90.0) |
| Ear and labyrinth | Ear discomfort | 0 | 1 (6.7) | 0 | 0 |
| disorders | Ear pain | 0 | 0 | 0 | 1 (10.0) |
| Endocrine disorders | Hypothyroidism | 0 | 1 (6.7) | 0 | 0 |
| Eye disorders | Blepharospasm | 0 | 0 | 1 (10.0) | 0 |
| | Diplopia | 0 | 1 (6.7) | 0 | 0 |
| | Dry eye | 0 | 1 (6.7) | 0 | 1 (10.0) |
| | Eye irritation | 1 (6.7) | 0 | 0 | 0 |
| | Eye pain | 0 | 1 (6.7) | 0 | 1 (10.0) |
| | Eye pruritus | 0 | 1 (6.7) | 0 | 0 |
| | Photophobia | 1 (6.7) | 0 | 0 | 1 (10.0) |
| | Photopsia | 1 (6.7) | 0 | 0 | 0 |
| | Punctate keratitis | 1 (6.7) | 0 | 0 | 0 |
| | Vision blurred | 1 (6.7) | 0 | 1 (10.0) | 0 |
| | Visual impairment | 1 (6.7) | 0 | 0 | 1 (10.0) |
| | Vitreous floaters | 1 (6.7) | 0 | 1 (10.0) | 0 |
| Gastrointestinal | Abdominal discomfort | 0 | 1 (6.7) | 1 (10.0) | 0 |
| disorders | Abdominal distension | 0 | 1 (6.7) | 0 | 0 |
| | Abdominal pain upper | 0 | 1 (6.7) | 0 | 0 |
| | Bowel movement | 0 | 1 (6.7) | 0 | 0 |
| | irregularity | | | | |
| | Constipation | 0 | 1 (6.7) | 0 | 0 |
| | Diarrhoea | 1 (6.7) | 0 | 1 (10.0) | 0 |
| | Dry mouth | 1 (6.7) | 0 | 0 | 0 |
| | Faeces soft | 1 (6.7) | 0 | 0 | 0 |
| | Lip swelling | 0 | 0 | 0 | 1 (10.0) |
| | Nausea | 0 | 1 (6.7) | 0 | 0 |
| | Tongue dry | 1 (6.7) | 0 | 0 | 0 |
| | Toothache | 0 | 0 | 0 | 1 (10.0) |
| General disorders | Death | 1 (6.7) | 0 | 0 | 0 |
| and administration | Inflammation | 0 | 0 | 0 | 1 (10.0) |
| site conditions | Pain | 2 (13.3) | 0 | 0 | 0 |
| | Pyrexia | 0 | 1 (6.7) | 0 | 0 |
| Infections and | Bronchitis | 0 | 0 | 0 | 1 (10.0) |
| infestations | Conjunctivitis | 1 (6.7) | 0 | 0 | 0 |
| | Ear infection | 0 | 0 | 1 (10.0) | 0 |
| | Gastroenteritis | 1 (6.7) | 0 | 1 (10.0) | 0 |
| | Gastroenteritis viral | 0 | 1 (6.7) | 0 | 0 |
| | Herpes zoster | 1 (6.7) | 0 | 0 | 0 |
| | Hordeolum | 0 | 1 (6.7) | 0 | ″0) |
| | Influenza | 0 | 1 (6.7) | 0 | 0 |
| | Laryngitis | 1 (6.7) | 0 | 0 | 0 |
| | Nasopharyngitis | 2 (13.3) | 3 (20.0) | 1 (10.0) | 5 (50.0) |
| | Pharyngitis | 1 (6.7) | 0 | 0 | 0 |
| | streptococcal | | | | |
| | Pneumonia | 0 | 0 | 1 (10.0) | 0 |
| Infections and | Sialoadenitis | 1 (6.7) | 0 | 0 | 0 |
| infestations (cont.) | Sinusitis | 1 (6.7) | 0 | 0 | 0 |
| | Skin infection | 0 | 0 | 0 | 1 (10.0) |
| | Tooth abscess | 0 | 1 (6.7) | 0 | 0 |
| | Upper respiratory | 1 (6.7) | 1 (6.7) | 2 (20.0) | 2 (20.0) |
| | tract infection | | | | |
| | Vulvovaginal | 0 | 1 (6.7) | 0 | 0 |
| | candidiasis | | | | |
| Injury, poisoning | Ankle fracture | 1 (6.7) | 0 | 0 | 0 |
| and procedural | Back injury | 0 | 0 | 0 | 1 (10.0) |
| complications | Cartilage injury | 1 (6.7) | 0 | 0 | 0 |
| | Conjunctival | 0 | 1 (6.7) | 0 | 0 |
| | laceration | | | | |
| | Face injury | 1 (6.7) | 0 | 0 | 0 |
| | Fall | 1 (6.7) | 1 (6.7) | 0 | 0 |
| | Hand fracture | 1 (6.7) | 0 | 0 | 0 |
| | Hypobarism | 0 | 1 (6.7) | 0 | 0 |
| | Muscle strain | 0 | 0 | 0 | 1 (10.0) |
| | Radius fracture | 1 (6.7) | 0 | 0 | 0 |
| | Subdural haematoma | 1 (6.7) | 0 | 0 | 0 |
| | Wrist fracture | 0 | 0 | 0 | 1 (10.0) |
| Investigations | Alanine aminotransferase | 1 (6.7) | 0 | 1 (10.0) | 0 |
| | increased | | | | |
| | Aspartate aminotransferase | 1 (6.7) | 0 | 1 (10.0) | 0 |
| | increased | | | | |
| | Blood cholesterol | 0 | 1 (6.7) | 0 | 0 |
| | increased | | | | |
| | Blood creatinine | 1 (6.7) | 0 | 0 | 0 |
| | increased | | | | |
| | Blood iron decreased | 0 | 1 (6.7) | 0 | 0 |
| | Blood pressure increased | 0 | 1 (6.7) | 0 | 0 |
| | Blood triglycerides | 1 (6.7) | 0 | 0 | 0 |
| | increased | | | | |
| | Electrocardiogram | 0 | 0 | 1 (10.0) | 0 |
| | abnormal | | | | |
| | Low density lipoprotein | 1 (6.7) | 0 | 0 | 0 |
| | increased | | | | |
| | Vitamin D decreased | 2 (13.3) | 1 (6.7) | 0 | 0 |
| Metabolism and | Vitamin B12 deficiency | 1 (6.7) | 0 | 0 | 0 |
| nutrition disorders | | | | | |
| Musculoskeletal | Arthralgia | 1 (6.7) | 1 (6.7) | 1 (10.0) | 1 (10.0) |
| and connective | Arthritis | 0 | 0 | 1 (10.0) | 0 |
| tissue disorders | Back pain | 0 | 1 (6.7) | 0 | 1 (10.0) |
| | Muscle spasms | 1 (6.7) | 0 | 0 | 1 (10.0) |
| | Musculoskeletal pain | 1 (6.7) | 1 (6.7) | 0 | 1 (10.0) |
| | Neck pain | 0 | 0 | 0 | 1 (10.0) |
| | Osteoporosis | 0 | 1 (6.7) | 0 | 0 |
| | Pain in extremity | 1 (6.7) | 2 (13.3) | 0 | 0 |
| | Pain in jaw | 0 | 0 | 0 | 1 (10.0) |
| | Plantar fasciitis | 1 (6.7) | 0 | 0 | 0 |
| | Rotator cuff syndrome | 0 | 1 (6.7) | 0 | 0 |
| | Sjogren's syndrome | 1 (6.7) | 0 | 0 | 0 |
| | Tendonitis | 1 (6.7) | 0 | 0 | 0 |
| Nervous system | Headache | 1 (6.7) | 2 (13.3) | 0 | 1 (10.0) |
| disorders | Intracranial pressure | 0 | 1 (6.7) | 0 | 0 |
| | increased | | | | |
| | Nerve compression | 0 | 0 | 0 | 1 (10.0) |
| | Neuralgia | 1 (6.7) | 0 | 0 | 0 |
| | Syncope | 0 | 1 (6.7) | 0 | 0 |
| | Thoracic outlet syndrome | 0 | 0 | 0 | 1 (10.0) |
| | Visual field defect | 0 | 0 | 1 (10.0) | 0 |
| Psychiatric | Abnormal dreams | 0 | 1 (6.7) | 0 | 0 |
| disorders | | | | | |
| Psychiatric | Depression | 1 (6.7) | 0 | 0 | 0 |
| disorders (cont.) | Insomnia | 1 (6.7) | 0 | 0 | 2 (20.0) |
| | Irritability | 0 | 0 | 0 | 1 (10.0) |
| | Obsessive-compulsive | 0 | 0 | 0 | 1 (10.0) |
| | disorder | | | | |
| Renal and urinary | Nephrolithiasis | 1 (6.7) | 0 | 1 (10.0) | 0 |
| disorders | | | | | |
| Reproductive system | Premenstrual syndrome | 0 | 1 (6.7) | 0 | 0 |
| and breast disorders | | | | | 0 |
| Respiratory, | Cough | 0 | 2 (13.3) | 0 | |
| thoracic and | Oropharyngeal pain | 0 | 3 (20.0) | 1 (10.0) | 0 |
| mediastinal | Pleurisy | 0 | 0 | 1 (10.0) | 0 |
| disorders | Sinus congestion | 0 | 1 (6.7) | 0 | 0 |
| Skin and | Alopecia | 2 (13.3) | 0 | 0 | 1 (10.0) |
| subcutaneous tissue | Dry skin | 0 | 1 (6.7) | 0 | 0 |
| disorders | Eczema | 0 | 0 | 1 (10.0) | 0 |
| | Erythema | 0 | 0 | 0 | 1 (10.0) |
| | Hair texture abnormal | 0 | 0 | 0 | 1 (10.0) |
| | Rash | 1 (6.7) | 0 | 1 (10.0) | 0 |
| | Xanthelasma | 0 | 1 (6.7) | 0 | 0 |
| Surgical and | Hysterectomy | 0 | 1 (6.7) | 0 | 0 |
| medical procedures | Jaw operation | 0 | 1 (6.7) | 0 | 0 |
| | Knee arthroplasty | 1 (6.7) | 0 | 0 | 0 |
| | Tooth extraction | 0 | 1 (6.7) | 0 | 0 |
| Vascular disorders | Hypertension | 0 | 1 (6.7) | 0 | 2 (20.0) |
| | Peripheral coldness | 0 | 0 | 0 | 1 (10.0) |

### Discussion

In this TEASE-1 study, gildeuretinol acetate met its prespecified primary efficacy endpoint, slowing the mean growth rate of atrophic retinal lesions relative to placebo and natural history. Including the natural history cases expanded the study population, providing a more accurate, truer estimate of the treatment effect. The growth rates of atrophic retinal lesions in the placebo and natural history cases were indistinguishable.

The estimated treatment effect ranged from approximately 20% to 30%, depending on the calculation method. The positive treatment effect when comparing the cross-over patients only (12 months of placebo vs. 12 months of treatment for the same patients) underscores gildeuretinol acetate's capacity to alter the clinical trajectory of Stargardt disease.

In conclusion, oral gildeuretinol acetate, taken once daily for up to 24 months, slowed the expansion of retinal atrophic lesions in individuals with Stargardt disease.
EXAMPLE 2: In individuals with asymptomatic Stargardt Disease, daily oral gildeuretinol acetate 14 mg capsule arrested loss of vision and retinal pathology.

### Methods

### Trial Design and Oversight

The tolerability and effects of ALK-001 (gildeuretinol acetate) in Stargardt Disease (Clinical trial group 3, also known as TEASE-3) study was an open-label, multicenter, case control study designed to evaluate the efficacy and safety of gildeuretinol acetate on the progression of Stargardt disease in asymptomatic individuals. The trial was conducted at multiple sites in the United States.

To assess the safety and efficacy of gildeuretinol acetate in preventing vision loss in asymptomatic individuals, gildeuretinol acetate 14 mg capsules were administered to individuals showing early signs of retinal damage while maintaining approximately 20/20 vision and no subjective loss of vision. Participants had a family history of Stargardt disease, particularly a two-year older sibling carrying identical genetic mutations and already exhibiting symptoms. Based on the above family history and clinical observations, the asymptomatic individuals were likely to develop retinal lesions, loss of photoreceptors, drops in retinal sensitivity, loss of visual acuity, indicating the onset of symptoms, i.e. a progression to the symptomatic phase, including vision loss within two years, mirroring the progression observed in their older siblings.

Testing individuals for Stargardt disease before they exhibit symptoms (presymptomatic testing) can result in unwarranted stigmatization and emotional stress. Testing asymptomatic individuals in the absence of a potential intervention is, therefore, controversial. All asymptomatic Stargardt disease patients were thus administered gildeuretinol acetate. Specific dose levels were not disclosed to the patients. Disease progression was compared to the symptomatic siblings with identical genetic mutations when aged-matched who had not received gildeuretinol acetate.

The study protocol was approved at each site by an independent institutional review board. The trial was conducted in accordance with the principles of the Declaration of Helsinki, International Conference on Harmonization E6 Good Clinical Practice Consolidated Guideline, and other relevant regulations. All participants provided written informed consent prior to screening or initiation of any study-related procedures and received visit-based reimbursement as approved by the institutional review boards. No additional stipends or incentives were provided.

### Patients

Patients 10 years of age and older, clinically diagnosed with Stargardt disease by a physician, diagnosis further supported by a genetic report indicating at least one ABCA4 mutation, exhibiting early signs of maculopathy, but with approximately 20/20 visual acuity and no complaints of visual impairment.

### Treatments

Patients received gildeuretinol acetate 14 mg capsules, to be taken orally once daily. Trial visits occurred every 6 months approximately following screening and treatment start visits.

### Study End Points and Assessments

Disease progression was monitored by best-corrected visual acuity (BCVA) reported in number of Early Treatment Diabetic Retinopathy Study (ETDRS) letters, fundus autofluorescence (FAF) imaging, loss of photoreceptors (as measured by the ellipsoid zone band loss) by optical coherence tomography (OCT) and retinal sensitivity by microperimetry.

### Safety

Ocular safety was assessed by retinal imaging and ocular exam, which included evaluation of the anterior and posterior segments and the lens. Systemic safety was assessed by physical exam, comprehensive clinical labs (biochemistry, hematology, lipids), and 12-lead ECG.

### Statistical Analyses

Measurements from the left and right eye for each patient were averaged for each visit to produce a single statistical unit for both eyes. For segmented linear regressions, knot times were selected so that three visits were included in the first segment.

### Results

### Participants

Three asymptomatic Stargardt disease patients, with 20/20 visual acuity, 13, 17, and 18 years of age, were enrolled. All three patients had signs of maculopathy, including one or multiple of the following Diagnostic Indicators: thinned outer nuclear layers, granular or missing ellipsoid zone layers, and parafoveal photoreceptor loss by OCT, and hyperautofluorescent patterns surrounding the foveal center by FAF imaging. Each enrolled patient had a symptomatic Stargardt disease sibling who was about two years older. The older untreated siblings had mild to moderate visual acuity loss, outer retinal atrophy in the foveal region, and a diffuse, mottled autofluorescent pattern with hyper-autofluorescent flecks. Genetic testing further supported that each sibling pair shared the same disease-causing mutations on the ABCA4 gene.

Between April 2017 and December 2019, the three patients took gildeuretinol acetate 14 mg oral capsules daily for two years. After the end of this initial two years of treatment, two patients continued treatment in an extension study and one participant discontinued treatment due to relocation. After the initial two years of treatment, each patient was the same age as when their older sibling started to lose vision. After the additional two years of treatment, corresponding to a total of four years of treatment with gildeuretinol acetate, the two treated patients were two years older than the age of onset of their symptomatic Stargardt disease sibling.

### Plasma Gildeuretinol

After the first two months of treatment, all three participants achieved an 80% replacement of plasma vitamin A (retinol) with gildeuretinol. Over 80% replacement was sustained throughout the treatment period.

### Best Corrected Visual Acuity

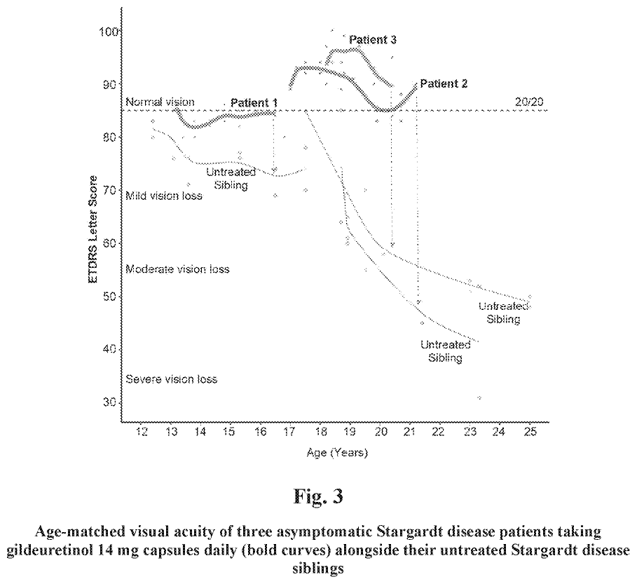

BCVA for the treated siblings remined stable at 88±4 ETDRS letters (~20/20), despite their untreated siblings having mild to moderate, uncorrectable BCVA of 66±10 ETDRS letters (between 20/40 and 20/100), corresponding to an average improvement of 22±13 letters or four lines of vision on the ETDRS eyechart (FIG. 3).

### Fundus Autofluorescence (FAF) Imaging

After two to four years of treatment, none of the three treated Stargardt disease patients exhibited changes by FAF: no retinal flecks appeared, no new areas of increased or decreased autofluorescence formed, and no new atrophy developed. In contrast, the three untreated Stargardt disease age-matched older siblings all showed year-over-year, progressive worsening by FAF imaging, including expansion of hyperautofluorescent rings, mottled areas, and development of atrophic retinal lesions.

### Photoreceptors Density by Optical Coherence Tomography

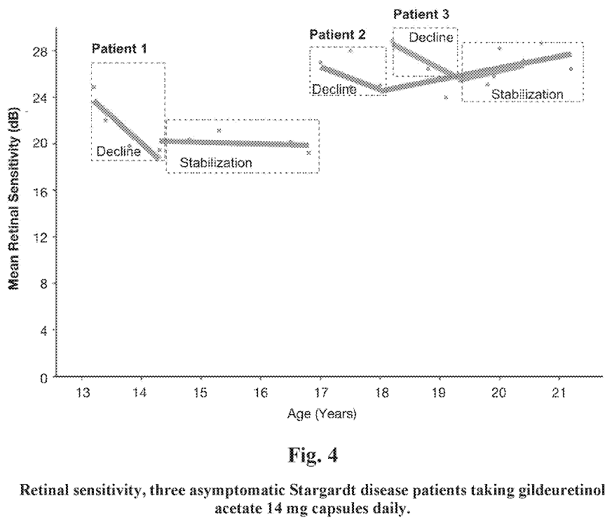

During the first six months of treatment, photoreceptor loss progressed, on average, by approximately 0.18 mm²/year for two treated patients, which was consistent with the natural history and the fact that the disease process had already started at treatment initiation. For the following years of treatment, photoreceptor loss trended toward an improvement (FIG. 4). For the third patient, no photoreceptors were lost during the full treatment period. The average area of photoreceptor loss was limited to 0.36±0.32 mm² or 20% of the foveal area, consistent with the maintenance of visual acuity.

### Retinal Sensitivity by Fundus-Tracking Microperimetry

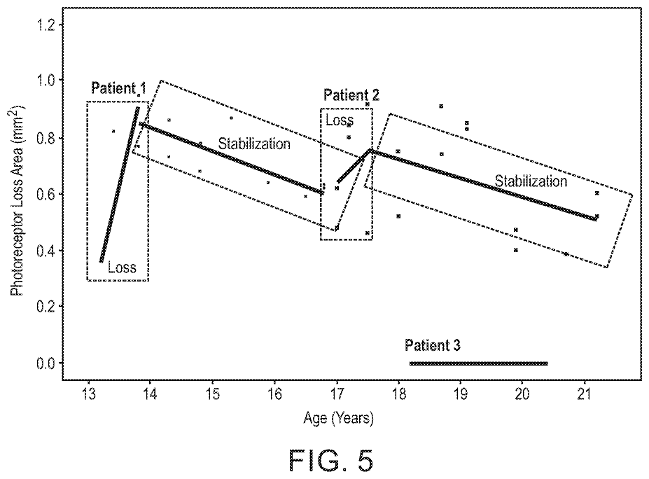

During the first year of treatment, mean retinal sensitivity decreased by an average of −3.1±1.5 dB/year. For the following years of treatment, mean retinal sensitivity trended toward an improvement with an average rate of retinal sensitivity improvement of +0.93±0.7 dB/year (FIG. 5).

### Safety

None of the three Stargardt disease asymptomatic patients exhibited adverse reactions linked to either excessive or insufficient vitamin A, such as delays in dark adaptation or night blindness.

### Discussion

In this TEASE-3 study, three asymptomatic Stargardt patients with a high likelihood of developing vision loss and symptoms of Stargardt disease in the next two years, based on their family history and clinical findings, took gildeuretinol acetate 14 mg oral capsules daily for 2 to 4 years. There was no observable disease progression during the treatment period. With 80% replacement of retinol (vitamin A) with gildeuretinol (deuterated vitamin A), the aberrant dimerization of vitamin A that leads to the formation of toxic vitamin A dimers and Stargardt disease is corrected to that of unaffected individuals.

In Stargardt disease, progressive FAF changes occur year-over-year. The absence of FAF changes in all three Stargardt disease patients treated with gildeuretinol acetate contrasts with the natural history of the disease and suggests a treatment-induced arrest, as supported by the stabilization of BCVA. The similar degree of retinal pathology by FAF in historical age-matched siblings with Stargardt disease when both are untreated, further emphasizes the significance of the complete absence of changes by FAF imaging.

Reported rates of photoreceptors lost (Ellipsoid Zone band loss) in individuals with Stargardt disease ranged from −0.33±0.38 to −1.20±1.29 mm²/year. (7-9) Simultaneously, retinal sensitivity is reported to decline at a rate of −2.2±3.0 dB/year. (11) During the first six months to a year of treatment, we observed similar declines in photoreceptor and retinal sensitivity. However, with prolonged treatment both outcomes trended toward an improvement. Previous evidence, TEASE-1, suggests that administering gildeuretinol acetate in later disease stages, slows disease progression; this work, TEASE-3, suggests early administration may arrest disease development.

For individuals with asymptomatic Stargardt Disease, once-daily oral gildeuretinol acetate, 14 mg capsules, taken for up to four years, effectively prevented vision loss and further retinal damage, in effect arresting disease progression for the duration of the intervention.

1-14. (canceled)

15. A method of delaying the onset of Stargardt disease symptoms in an asymptomatic Stargardt disease patient, comprising administering to the patient an effective amount of gildeuretinol acetate.

16. The method of claim 15, wherein the patient has a Stargardt Associated Mutation.

17. The method of claim 15, wherein the patient has biallelic Stargardt Associated Mutations.

18. The method of claim 15, wherein the patient has an ABCA4 mutation.

19. The method of claim 15, wherein the patient has retinal atrophic lesions, retinal flecks, pigment migration, retinal thinning, retinal atrophy, areas of hypo or hyper-autofluorescence, ellipsoid zone loss, abnormal or thickened external limiting membrane, photoreceptor loss, or maculopathy.

20. The method of claim 15, wherein the Stargardt disease symptom is vision loss.

21. The method of claim 15, comprising administering the gildeuretinol daily for a period of 2 years.

22. The method of claim 15, comprising administering the gildeuretinol daily for a period of 4 years.

23. The method of claim 21, wherein no visible disease progression by fundus autofluorescence imaging occurs in the patient during the period of administration.

24. The method of claim 21, wherein vision loss does not occur in the patient during the period of administration.

25. A method of delaying the onset of vision loss in an asymptomatic Stargardt disease patient, comprising administering to the patient an effective amount of gildeuretinol acetate.

26. The method of claim 25, wherein the patient has a Stargardt Associated Mutation.

27. The method of claim 25, wherein the patient has biallelic Stargardt Associated Mutations.

28. The method of claim 25, wherein the patient has an ABCA4 mutation.

29. The method of claim 25, wherein the patient has retinal atrophic lesions, retinal flecks, pigment migration, retinal thinning, retinal atrophy, areas of hypo or hyper-autofluorescence, ellipsoid zone loss, abnormal or thickened external limiting membrane, photoreceptor loss, or maculopathy.

30. The method of claim 25, wherein the vision loss is characterized by one or more features selected from: loss of central vision, flashes of light, blurred vision, difficulty in seeing in dim lighting or at night, difficulty in distinguishing colors, difficulty in adjusting to bright light exposure, difficulty in adjusting to the dark, difficulty in tracking moving objects, difficulty recognizing faces, difficulty writing, difficulty reading, and blind spots in the field of vision.

31. The method of claim 25, comprising administering the gildeuretinol daily for a period of 2 years.

32. The method of claim 25, comprising administering the gildeuretinol daily for a period of 4 years.

33. The method of claim 31, wherein no visible disease progression by fundus autofluorescence imaging occurs in the patient during the period of administration.

34. The method of claim 31, wherein vision loss does not occur in the patient during the period of administration.

35. A method of treating a Stargardt disease patient to delay the onset of vision loss in the Stargardt disease patient, wherein treatment is initiated prior to the patient experiencing vision loss resulting from the Stargardt disease, comprising administering to the patient an effective amount of gildeuretinol acetate.

36. The method of claim 35, wherein the patient has a Stargardt Associated Mutation.

37. The method of claim 35, wherein the patient has biallelic Stargardt Associated Mutations.

38. The method of claim 35, wherein the patient has an ABCA4 mutation.

39. The method of claim 35, wherein the patient has retinal atrophic lesions, retinal flecks, pigment migration, retinal thinning, retinal atrophy, areas of hypo or hyper-autofluorescence, ellipsoid zone loss, abnormal or thickened external limiting membrane, photoreceptor loss, or maculopathy.

40. The method of claim 35, wherein the vision loss is characterized by one or more features selected from: loss of central vision, flashes of light, blurred vision, difficulty in seeing in dim lighting or at night, difficulty in distinguishing colors, difficulty in adjusting to bright light exposure, difficulty in adjusting to the dark, difficulty in tracking moving objects, difficulty recognizing faces, difficulty writing, difficulty reading, and blind spots in the field of vision.

41. The method of claim 35, comprising administering the gildeuretinol daily for a period of 2 years.

42. The method of claim 35, comprising administering the gildeuretinol daily for a period of 4 years.

43. The method of claim 41, wherein no visible disease progression in the patient by fundus autofluorescence imaging occurs during the period of administration.

44. The method of claim 41, wherein vision loss does not occur in the patient during the period of administration.
